(19) United States
(12) Patent Application Publication
(10) Pub. No.: US 2025/0302769 A1
(43) Pub. Date: Oct. 2, 2025

(54) SELF-EMBEDDING SILVER NANOPARTICLE BIOMASS WASTE COMPOSITIONS

(71) Applicant: The United States of America, as represented by the Secretary of Agriculture, Washington, DC (US)

(72) Inventors: SUNGHYUN NAM, METAIRIE, LA (US); MICHAEL W. EASSON, NEW ORLEANS, LA (US); JACOBS H. JORDAN, DESTREHAN, LA (US); ZHONGQI HE, NEW ORLEANS, LA (US)

(21) Appl. No.: 19/076,773

(22) Filed: Mar. 11, 2025

Related U.S. Application Data
(60) Provisional application No. 63/571,101, filed on Mar. 28, 2024.

(51) Int. Cl. A61K 9/70; A01N 59/16; A01P 1/00; A01P 3/00; A61K 9/14; A61K 33/38; A61P 31/02

(52) U.S. Cl. CPC A61K 9/70; A01N 59/16; A01P 1/00; A01P 3/00; A61K 9/14; A61K 33/38; A61P 31/02

(57) ABSTRACT

Compositions and methods of making and using silver nanoparticles embedded in biomass waste matrixes of various types is described. Exemplified compositions include a silver nanoparticle embedded in a cotton gin waste nanofiber composite. Compositions and methods of making and using aerogels comprising silver nanoparticles in cotton gin waste nanofiber are described. Exemplified uses of compositions include use as antimicrobial agents.

### CROSS-REFERENCE TO RELATED APPLICATIONS

This application claims the benefit of U.S. Provisional Application No. 63/571,101, titled “SELF-EMBEDDING SILVER NANOPARTICLE BIOMASS WASTE COMPOSITIONS” filed Mar. 28, 2024, which is incorporated herein by reference in its entirety.

### FIELD OF THE INVENTION

The inventions herein relate to novel biomass waste-derived silver nanoparticle embedded compositions, their synthesis and methods of using the compositions.

### BACKGROUND OF THE INVENTION

Cotton gin waste or cotton gin trash (CGT) is a byproduct (the complex mixture of burrs, sticks, motes, and other particles) generated during the cotton ginning process, where cotton fibers are separated from the seed bolls. Conventional practices for the disposal of cotton gin waste include landfilling and composting for soil amendment. However, these methods are costly and not always suitable for all climatic conditions. Additionally, CGT can be used as cattle feed, but its low protein content poses limitations. The estimated disposal cost of CGT is roughly $10 million a year. Additionally, over 1.7 million tons of gin trash are produced annually. As a result, this significant agro-industrial waste has been a major issue for the cotton ginning industry.

CGT is an excellent source of lignocellulose. Over the past decades, CGT has gained the attention for production of new materials. Current research exists to use CGT for the fabrication of polymer composites, insulation packaging, particleboard, masonry blocks, adsorbent materials, etc.

Other important sources of agricultural biomass or plant biomass waste are exemplified by cotton gin motes, cotton seed hulls, rice straw, sugar cane bagasse, etc. Cotton gin motes are immature cotton seeds surrounded by entangled immature cotton fibers and are a biomass product that results from cotton ginning operations. Cotton seed hulls are the outer covering of cotton seeds comprising the seed hull and lint and are the agricultural by-product from the extraction of cottonseed oil. Waste corrugated cardboard is a fibrous paper product made up of layered heavy papers used primarily in the packaging industry. Rice straw is an agricultural by-product obtained from harvesting rice paddy and separating the grains from the rice plant. Sugarcane bagasse is the residual, dried, fibrous material that results from extracting the juice of sugarcane or sorghum stalks in the sugar processing industry.

There is a long-felt need for low-cost and zero-waste solutions that transform biomass waste such as cotton gin waste into value-added products through innovative technology.

Compositions and methods described herein address some of these important problems.

### SUMMARY OF THE INVENTION

Described herein are compositions and methods using cotton gin waste to synthesize silver nanoparticle embedded cotton gin waste nanofibers and aerogels.

In an aspect, compositions and methods herein describe silver nanoparticle-embedded cotton gin waste nanofiber compositions with a concentration of silver of 0.1-500,000 mg/kg is described. The concentration of silver nanoparticles in the embedded cotton gin waste CNF is about 7 weight percent in one aspect.

The silver nanoparticle-embedded cotton gin waste nanofibers have silver nanoparticles having an average size of about 18 nm in some embodiments. The average diameter of NPs determined from the TEM images is from 6-15 nm in some embodiments. The diameter is about 10.2±3.4 nm in some embodiments. Other embodiments have diameters ranging in size from 1-35 nm. The silver nanoparticle-embedded cotton gin waste nanofibers (Ag-CNF) exhibit a strong surface plasmon resonance peak centered at 423 nm.

In one aspect, silver nanoparticle-embedded nanofibers are prepared by extracting cellulose nanofiber (CNF) from cotton gin waste (or trash) (CGT) through a mechanical process and embedding antimicrobial silver nanoparticles (Ag NPs) directly into the CNF matrix.

In some embodiments, nested nanostructure silver nanoparticle-embedded cotton gin waste nanofibers (Ag-CNF) compositions provide maximized antimicrobial activity through a stable and highly reactive surface. The compositions are useful in various embodiments in applications requiring potent antimicrobial effects, such as in medical devices, wound dressings, and food packaging materials.

In various embodiments, the silver nanoparticle-embedded cotton gin waste nanofiber compositions (Ag-CNF) exhibit potent bacterial reduction activity ranging from 0.3 CFU/mL to about 7.0 Log CFU/mL. Exemplified reductions for compositions are about 5.4 Log CFU/mL for P. aeruginosa and >4.6 Log CFU/mL for S. aureus in 24 h and 4.1 Log CFU/mL for P. aeruginosa and 0.7 CFU/mL for S. aureus in 1 hour, corresponding to percent reductions of over 99.99% and 79.36%, respectively. At 24 h, Ag-CNF inactivation is exemplified by levels exceeding 6.6 Log CFU/mL for both S. Typhimurium and L. monocytogenes. Within just a 10 min-exposure, Ag-CNF shows reductions exemplified by >4.9 Log CFU/mL for S. Typhimurium and >5.5 Log CFU/mL for L. monocytogenes.

### BRIEF DESCRIPTION OF THE DRAWINGS

FIG. 1 shows UV-vis absorbance spectra of CGT, CNF and Ag-CNF.

FIG. 2 shows TEM images of CNF at (A) low and (B) high magnifications.

FIG. 3 shows TEM images of Ag-CNF at (A) low and (B) high magnifications.

FIG. 4 shows the size distribution histogram of Ag NPs formed within CNFs.

FIG. 5 shows EDS spectrum of CNF.

FIG. 6 shows EDS spectrum of Ag-CNF.

FIG. 7 shows UV-vis spectra of CNF suspension and Ag-CNF suspension and their photographs.

FIG. 8 shows (A) Minimum inhibitory concentrations (MICs) of Ag-CNF against P. aeruginosa and S. aureus. (B) Log reductions in P. aeruginosa and S. aureus populations following exposure to CNF and Ag-CNF for 1 h and 24 h. nr denotes no reduction. Numbers atop each bar represent percent reductions.

FIG. 9 shows photographs of inhibition zones for CNF (top) and Ag-CNF (bottom) for (A) S. Typhimurium and (B) L. monocytogenes.

FIG. 10 shows Log reductions in S. Typhimurium and L. monocytogenes populations following exposure to CNF and Ag-CNF for 10 min and 24 h. Numbers atop each bar represent percent reductions

FIG. 11 shows SEM images of S. Typhimurium cells (A) unexposed, (B) exposed to CNF for 24 h, and (C) exposed to Ag-CNF for 24 h.

FIG. 12 shows SEM images of L. monocytogenes cells (A) unexposed, (B) exposed to CNF for 24 h, and (C) exposed to Ag-CNF for 24 h.

FIG. 13 shows SEM images of CNF at (A) low and (B) high magnifications.

FIG. 14 shows SEM images of Ag-CNF at (A) low and (B) high magnifications.

FIG. 15 shows the surface areas of CNF aerogel and Ag-CNF aerogel.

FIG. 16 shows pore volumes of CNF aerogel and Ag-CNF aerogel.

FIG. 17 shows XRD patterns of CNF (bottom) and Ag-CNF (top).

FIG. 18 shows UV-vis spectra of various biomass microparticles (A) before and (B) after in situ synthesis of Ag NPs.

FIG. 19 shows photographic images of control cardboard particles (left) and Ag NP-cardboard particles

FIG. 20 shows photographic images of control cotton gin mote particles (left) and Ag NP-cotton gin mote particles.

FIG. 21 shows photographic images of control cottonseed hulls particles (left) and Ag NP-cotton gin mote particles.

FIG. 22 shows photographic images of control rice straw particles (left) and Ag NP-rice straw particles.

FIG. 23 shows photographic images of control sugarcane bagasse particles (left) and Ag NP-sugarcane bagasse particles.

FIG. 24 shows photographic images of control pine wood particles (left) and Ag NP-pinewood particles.

FIG. 25 shows scanning electron microscopic (SEM) images of control cardboard particles (top) and Ag NP-cardboard particles at 50,000×.

FIG. 26 shows scanning electron microscopic (SEM) images of cotton gin motes particles (top) and Ag NP-cotton gin motes particles at 50,000×.

FIG. 27 shows scanning electron microscopic (SEM) images of cotton seed hulls particles (top) and Ag NP-cotton seed hulls particles at 50,000×.

FIG. 28 shows scanning electron microscopic (SEM) images of rice straw particles (top) and Ag NP-rice straw particles at 50,000×.

FIG. 29 shows scanning electron microscopic (SEM) images of sugar cane bagasse particles (top) and Ag NP-sugar cane bagasse particles at 50,000×.

FIG. 30 shows scanning electron microscopic (SEM) images of pine wood particles (top) and Ag NP-pine wood particles at 50,000×.

FIG. 31 shows the energy-dispersive X-ray spectrum (EDS) of Ag NP-cardboard particles.

FIG. 32 shows the energy-dispersive X-ray spectrum (EDS) of Ag NP-cotton gin motes particles.

FIG. 33 shows the energy-dispersive X-ray spectrum (EDS) of Ag NP-cotton seed hulls particles.

FIG. 34 shows the energy-dispersive X-ray spectrum (EDS) of Ag NP-rice straw particles.

FIG. 35 shows the energy-dispersive X-ray spectrum (EDS) of Ag NP-sugar cane bagasse particles.

FIG. 36 shows the energy-dispersive X-ray spectrum (EDS) of Ag NP-pine wood particles.

FIG. 37 shows the concentration of silver in various biomass microparticles after the treatment.

### DETAILED DESCRIPTION

Described herein are silver nanoparticle-embedded cotton gin waste nanofiber compositions with antimicrobial activity. The concentration of embedded silver in the compositions ranges between 0.1-500000 mg/kg. The average concentration of silver in the composition is about 18.7 wt % in some embodiments.

The silver nanoparticle-embedded cotton gin waste nanofibers have silver nanoparticles having a particle size range between 1 nm and 85 nm embedded in the cotton gin waste nanofiber matrix. In some embodiments, the silver nanoparticles embedded have an average size of about 18 nm.

In one embodiment, the silver nanoparticle-embedded cotton gin waste nanofibers have a concentration of silver of 187,000±67000 mg/kg (18.7±6.7 wt %). In one embodiment, the silver nanoparticle-embedded cotton gin waste nanofibers have a concentration of silver of 190,000 mg/kg.

In one embodiment, the silver nanoparticles embedded in the cotton gin waste nanofiber described in various embodiments herein show even distribution or dispersion on the cotton gin waste nanofiber resulting in no agglomeration and aggregation of nanoparticles. In one embodiment, the silver nanoparticles embedded in the cotton gin waste nanofiber described in various embodiments herein show even distribution or dispersion on the cotton gin waste nanofiber resulting in minimal agglomeration and aggregation of nanoparticles. Agglomeration and aggregation can negatively affect their surface area and often their nanoscale properties.

Also described herein are methods of producing silver nanoparticle-embedded cotton gin waste nanofiber compositions comprising the steps of a) preparing a cotton gin waste nanofiber and b) treatment of cotton gin waste nanofiber with a silver precursor, and c) optionally washing to remove unreacted silver precursor to form said silver nanoparticle-embedded cotton gin waste nanofiber.

In one aspect, cotton gin waste nanofiber is produced by treatment of cotton gin waste nanofiber in an aqueous silver solution at an elevated temperature.

In one aspect, cotton gin waste nanofiber is produced by leveraging the unique capability of CNF as a reagent-free, active bioplatform for the in-situ synthesis of Ag NPs. The nanofiber produced individual, separated NPs without agglomeration within the CNF matrix, enhancing surface reactivity for antimicrobial performance.

In one aspect, a method of treating a microorganism with a composition is described comprising Ag NP-embedded CNF by an antimicrobial mechanism involving ionization rather than nanomechanical attack. In one aspect, a method of treating a microorganism with a composition is described comprising Ag NP-embedded CNF by effective and sustained release of Ag ions for antimicrobial activity.

In one aspect the Ag NP-embedded CNF is used for wound dressings. In one aspect the Ag NP-embedded CNF are used for food safety. In one aspect the Ag NP-embedded CNF are used for wound dressings. In one aspect the Ag NP-embedded CNF are used for agricultural disease management. In one aspect, the Ag NP-embedded CNF compositions are used to address agricultural waste and environmental waste challenges by transforming agricultural byproducts into high-performance functional materials.

In one aspect, cotton gin waste nanofiber is produced by processing using one or more steps of a high-pressure homogenizer, an ultrafine grinder and/or a microfluidizer. Processing is repeated for any of the steps according to the property of the product desired as necessary.

In one embodiment, the cotton gin waste nanofiber is produced by the steps of: grinding of cotton gin waste in a mill with about a 20-80 mesh sieve, treating the obtained powder sample with a NaOH solution (about 4% solution, for example) to remove extractives, washing with water, suspending the alkali-treated cotton gin waste in water and then homogenizing for example by high-shear mixing, then homogenizing the lignocellulose slurry with high-shearing forces using a high-pressure homogenizer, pumping through a ceramic interaction chamber up to five times or more as needed (for example, with a 200 μm chamber), pumping through diamond Z-shaped interaction chamber (for example, about a 87 μm chamber) added in series with a second ceramic interaction chamber (for example, about a 200 μm chamber) optionally for an additional of five passes or more as needed with operating pressure set around about 210 MPa.

Methods of producing silver nanoparticle-embedded cotton gin waste nanofiber are described herein in different embodiments. In one embodiment the steps of the method include: grinding cotton gin waste to a powder, for example, in a mill with about a 20 to 80 mesh sieve; treating the obtained powder sample with a base, for example with a 4 wt % NaOH solution to remove extractives; washing with water; suspending the alkali-treated cotton gin waste in water and then high-shear mixing; homogenizing and fibrillating the lignocellulose slurry with high-shearing forces using, for example, a high-pressure homogenizer, a microfluidics interaction chamber, an ultrafine grinder such as a supermasscolloider, or ultrasonication; in one example the slurry is pumped through an interaction chamber at high pressure of about 200-3000 bar where the slurry particle size is reduced by impaction and mixing; in a second embodiment the sample is forced through a narrow aperture through a z- or y-shaped interaction chamber where the narrow channel width and abrupt change in flow creates cavitation and shear reducing particle size; in one embodiment the slurry is mixed with high shearing forces between two non-porous discs at very low (under 4 μm) clearance; in another embodiment the lignocellulose slurry is subjected to ultrasonication which mixes and defibrillates the fibers. In each embodiment the slurry is continuously passed through the method of preparation and the size of the chamber or clearance is reduced with successive passes until the desired sample consistency is reached; and heat treatment of cotton gin waste nanofiber with a silver precursor to form the silver nanoparticle-embedded cotton gin waste nanofiber. Heat treatment could be using a hot plate or flame heating, microwave irradiation, oven heating, infrared heating, induction heating etc. In one embodiment, the reaction form could be a mixture of nanofiber suspension and an aqueous silver precursor solution and saturated nanofibers with an aqueous silver precursor solution. In other embodiments, the silver nanoparticle-embedded cotton gin waste nanofiber was formed by heating a mixture of nanofiber suspension and an aqueous silver precursor solution using a hot plate.

Silver nanoparticle-embedded cotton gin waste nanofiber aerogel compositions are described. The silver nanoparticle aerogel in one embodiment has a pore volume of 1 to 100 mm³/g. The silver nanoparticle aerogel has a pore volume of 30 mm³/g in other embodiments. The silver nanoparticle aerogel has a surface area of 1 to 100 m²/g in one embodiment. The silver nanoparticle aerogel has a surface area of about 9 m²/g.

Methods of treating a microbe by application of an effective amount of a composition comprising a silver nanoparticle-embedded cotton gin waste nanofiber. Some embodiments of the method treat a microbe which is a pathogenic bacterium. Some embodiments of the method treat a microbe which is a pathogenic fungus. Some embodiments of the method treat a surface selected from the group consisting of human skin, plant skin, animal skin, or a device surface. Some embodiments of the method treat a surface with a concentration of silver nanoparticle within the nanofibers between 0.1-500,000 mg/kg. Some embodiments of the method treat a surface with a concentration of silver nanoparticle within the nanofibers is between 1-25 wt %. Some embodiments of the method treat a surface with a concentration of silver nanoparticle within the nanofibers is between 5-20 wt %. Some embodiments of the method treat a surface with a concentration of silver nanoparticle within the nanofibers is between 5-12 wt %. Some embodiments of the method treat a surface with a concentration of silver nanoparticle within the nanofibers is about 7 wt %. Some embodiments of the method treat a microbe with a concentration of silver nanoparticle within the nanofibers is between 1-25 wt %. Some embodiments of the method treat a microbe with a concentration of silver nanoparticle within the nanofibers is between 5-20 wt %. Some embodiments of the method treat a microbe with a concentration of silver nanoparticle within the nanofibers is between 5-12 wt %. Some embodiments of the method treat a microbe with a concentration of silver nanoparticle within the nanofibers is about 7 wt %.

The aqueous silver precursor solution to form the silver nanoparticle-embedded cotton gin waste nanofiber is preferably a silver salt for example, silver nitrate. Other silver solutions that may be but are not limited to silver chloride, silver acetate, and other silver salts containing organic or inorganic anions.

Also described herein is a silver nanoparticle-embedded cotton gin waste nanofiber aerogel composition. The silver nanoparticle-embedded aerogel has a pore volume between 1 to 100 mm³/g.

In one embodiment, the silver nanoparticle-embedded aerogel has a pore volume of 30 mm³/g

In one embodiment, the silver nanoparticle-embedded aerogel has a surface area between 1 to 100 m²/g.

In one embodiment, the silver nanoparticle-embedded aerogel has a surface area of about 9 m²/g.

In one embodiment, the silver nanoparticle-embedded cotton gin waste nanofibers produce aerogel that has antimicrobial properties. In some embodiments, uses of silver nanoparticle-embedded cotton gin waste nanofibers include biodegradable packaging, food packaging, paper, textiles, composite materials, membranes, water filters, air filters, coatings, films, adhesives, personal care products, wound dressings, adsorbent materials, tissue engineering scaffolds, agriculture and horticulture products, plant growth substrates, soil amendments, crop disease controlling products, insecticidal spraying and bait products, pharmacological applications, etc., which have antibacterial, antifungal, and antiviral properties.

In one embodiment, silver nanoparticles can be embedded in other forms of cellulosic biomass (besides cotton gin waste), such as sugar cane bagasse, cotton seed hulls, rice straw, fruit peels, or water hyacinth.

In one embodiment, silver nanoparticles can be embedded in other forms of cotton gin waste (besides nanofiber), such as ground particles for similar applications.

Described herein are methods of treating a microbe with an effective amount of a composition comprising a silver nanoparticle-embedded cotton gin waste nanofiber. Also described herein are methods of treating a microbe with an effective concentration of silver nanoparticles in a matrix of cotton gin waste nanofiber.

In one embodiment, the application of a silver nanoparticle-embedded cotton gin waste nanofiber results in microbial growth inhibition. In one embodiment, the application of a silver nanoparticle-embedded cotton gin waste nanofiber results in growth inhibition of a pathogenic bacterium. In one embodiment, the application of a silver nanoparticle-embedded cotton gin waste nanofiber results in growth inhibition of pathogenic bacteria such as gram-positive Staphylococcus aureus ATCC 6538. In one embodiment, the application of a silver nanoparticle-embedded cotton gin waste nanofiber results in growth inhibition of pathogenic bacteria is gram-negative Pseudomonas aeruginosa (P. aeruginosa) ATCC 9027.

In one embodiment, the application of a silver nanoparticle-embedded cotton gin waste nanofiber results in growth inhibition of a pathogenic fungus.

In one embodiment, using the compositions described herein microbes can be inhibited which are found in various microbial growth environments including humans, animals, plants, device and tool surfaces, cleaning surfaces, domestic, industrial or medical work surfaces and the like that can be a substrate for microbial contamination, growth or transmission.

In one embodiment, the method of treatment is an application of an effective amount of a composition comprising a silver nanoparticle embedded in a matrix of cotton gin waste nanofiber with a concentration of 1.0-500,000 mg/kg silver nanoparticles.

The present disclosure provides a method of producing silver nanoparticles embedded in a matrix of cotton gin waste nanofibers by the steps of a) preparing a cotton gin waste nanofiber and b) treatment of cotton gin waste nanofiber in an aqueous silver precursor solution (optionally at an elevated temperature) and washing the nanofibers to form said silver nanoparticles (Ag NPs) within cotton gin waste nanofiber.

The method above additionally provides a process for cotton gin waste to self-produce and self-embed silver nanoparticles wherein cotton gin waste does not require the use of external reducing and stabilizing agents or dispersion processes for the silver nanoparticles.

In one embodiment the cotton gin waste may be partially delignified to form delignified pulp fibers which are passed through a high-pressure homogenizer, an ultrafine grinder or a microfluidizer to form nanocellulose. In preferred embodiments, delignification is not necessary, typically only the water/alkali soluble extractives are removed for processing purposes.

In one embodiment the cotton gin waste nanofibers—described herein have dimensions of 1 nm to 50 nm in diameter. In one embodiment the cotton gin waste nanofibers described herein have dimensions of 3 nm to 15 nm in width. In one embodiment the cotton gin waste nanofibers described herein have dimensions of 50 nm to 700 nm width. In one embodiment the cotton gin waste nanofibers described herein have dimensions of 25 nm to 500 nm in length. In one embodiment the cotton gin waste nanofibers described herein have dimensions of 500 nm to 5 μm in length. In one embodiment the cotton gin waste nanofibers described herein have dimensions of 5 μm to 500 μm in length.

In one embodiment the silver nanoparticles described herein have dimensions of 1nm to 50 nm in diameter. In one embodiment the silver nanoparticles described herein have dimensions of 3 nm to 15 nm in width. In one embodiment the silver nanoparticles described herein have dimensions of 5 nm to 25 nm in width. In one embodiment the silver nanoparticles described herein have dimensions of 50 nm to 700 nm width. In one embodiment the nanoparticles described herein have dimensions of 25 nm to 500 nm in length. In one embodiment the nanoparticles described herein have dimensions of 500 nm to 5 μm in length. In one embodiment the nanoparticles described herein have dimensions of 5 μm to 500 μm in length.

Plant derived biomass waste particles are derived from an abundance of post-harvest or post-production processes from agricultural biomass. Plant derived biomass waste particles include biomass from woody and non-woody plants and portions of the plant such as trunk, bark, leaf, stick, grass, stem, fruit, shell, hull, stalk, seed or root, etc. Plant derived biomass is dried and cut, ground, crushed, milled, or shaved to sizes between 0.1 mm and 2 cm. Dried plant derived biomass is processed by crushing, grinding or milling to sizes between 0.5 mm and 0.5 cm. Ground, milled or crushed plant biomass is milled using an ultrafine grinder or knife mil to a particle size sufficient to pass through a 20 to 80 mesh sieve having a particle size of less than 0.1 mm to less than 1.0 mm.

In one aspect a silver nanoparticle-embedded plant derived biomass waste nanofiber composite composition with a concentration of silver of 0.1-500,000 mg/kg is described. In different embodiments, the silver nanoparticle-embedded plant derived biomass waste nanofiber composite composition can be made from biomass waste such as cotton gin waste, cardboard, cotton gin motes, cotton seed hulls, rice straw, sugar cane bagasse or pine wood or mixtures thereof.

In one aspect, the silver nanoparticle-embedded plant derived biomass waste particle has a concentration of silver is between 0.1 and 30 wt %. In one aspect, the silver nanoparticle-embedded plant derived biomass waste particle has a concentration of silver is between 1 and 15 wt %. In one aspect, the silver nanoparticle-embedded plant derived biomass waste particle has a concentration of silver is between 1-7 wt %. In one aspect, the silver nanoparticle-embedded plant derived biomass waste particle has a concentration of silver is between 1.6 and 5.9%.

In one aspect, a method of treating a microbe by application of an effective amount of a composition containing silver nanoparticle-embedded plant derived biomass waste particle is described. In one aspect, the method uses a biomass waste particle which is cotton gin mote waste particle.

In one aspect, the method uses a biomass waste particle which is a cardboard waste particle. In one aspect, the method uses a biomass waste particle which is a cotton seed hulls particle. In one aspect, the method uses a biomass waste particle which is a sugar cane bagasse particle. In one aspect, the method uses a biomass waste particle which is a rice straw particle. In one aspect, the method uses a biomass waste particle which is a pine wood particle.

In one aspect, a methods of preparing silver nanoparticle-embedded plant derived biomass waste particle compositions by the steps of: grinding the biomass waste material such as cotton gin waste, cardboard, cotton gin motes, cotton seed hulls, rice straw, sugar cane bagasse or pine wood or mixtures thereof with a 20-mesh sieve; suspending the particles in an aqueous AgNO₃ solution and optionally heating; optionally rinsing the particles with water and separation of particles by filtration. The steps above may also be executed with an aqueous alkaline solution at room temperature combined with the AgNO₃ and optionally heating.

In one embodiment, a silver nanoparticle-embedded cellulosic biomass nanocomposite with a concentration of silver of 0.1-500,000 mg/kg is described.

In one embodiment, the silver nanoparticle-embedded cellulosic biomass nanofiber composite is derived from a biomass source which is cotton gin waste.

In one embodiment, the silver nanoparticle-embedded cotton gin waste nanofiber composite has a concentration of silver is between 0.1-30 wt %.

In one embodiment, the silver nanoparticle-embedded cotton gin waste nanofiber has a concentration of silver of about 19 wt %.

In one embodiment, the silver nanoparticle-embedded cotton gin waste nanofiber composite has a particle size between 1 nm and 150 nm in the cotton gin waste nanofiber matrix.

In one embodiment, the silver nanoparticle-embedded cotton gin waste nanofiber composite with silver nanoparticles having size of between 3 nm and 50 nm.

In one embodiment, the silver nanoparticle-embedded cotton gin waste nanofiber composite of with silver nanoparticles having average size of about 18 nm.

In one embodiment, the silver nanoparticle-embedded cotton gin waste nanofiber composite with silver nanoparticles having uniformity in size and distribution in the cotton gin waste nanofiber.

In one embodiment, a method of producing silver nanoparticle-embedded cotton gin waste nanofiber composite is described comprising the steps of:
a) preparing a cotton gin waste nanofiber,
b) treating of cotton gin waste nanofiber suspension with an aqueous silver precursor solution to form the silver nanoparticle-embedded cotton gin waste nanofiber.

In one embodiment, the cotton gin waste nanofiber composite is produced by the steps of:
grinding cotton gin waste in a mill to a size of 20 to 80 mesh sieve,
treating the obtained powder sample with a fluid to remove extractives,
washing with water,
suspending the alkali-treated cotton gin waste in water and then high-shear mixing,
homogenizing the lignocellulose slurry with high-shearing forces using a high-pressure homogenizer,
pumping through a 200-1000 μm interaction chamber five times,
pumping through a second 50-500 μm interaction chamber with a narrower aperture added in series with a 200-1000 μm interaction chamber for one or more passes with operating pressure set to about 200-300 MPa.

In one embodiment, the operating pressure for the method is set to about 210 MPa.

In one embodiment, the aqueous silver precursor solution comprises silver nitrate.

In one embodiment, the method employs washing to remove unreacted silver nitrate precursor to form said silver nanoparticle-embedded cotton gin waste nanofiber.

In one embodiment, the interaction chamber is Z-shaped and about 20-200 μm.

In one embodiment, the Z-shaped interaction chamber is about 90 μm.

In one embodiment, the fluid to remove extractives is alkaline.

In one embodiment, the fluid to remove extractives is an aqueous NaOH solution.

In one embodiment, a silver nanoparticle-embedded cotton gin waste nanofiber aerogel composition is described.

In one embodiment, the concentration of silver nanoparticles of 0.1-500,000 mg/kg in the aerogel composition.

In one embodiment, the silver nanoparticle aerogel has a pore volume of between about 1 to 100 mm³/g

In one embodiment, the silver nanoparticle aerogel has a pore volume of 30 mm³/g

In one embodiment, the silver nanoparticle aerogel has a surface area of 1 to 100 m²/g.

In one embodiment, the silver nanoparticle aerogel has a surface area of about 9 m²/g.

In one embodiment, a method of treating a microbe by application of an effective amount of a composition comprising a silver nanoparticle-embedded cotton gin waste nanofiber composite is described.

In one embodiment, the treating with an effective amount of silver nanoparticle-embedded cotton gin waste nanofiber composite results in microbial growth inhibition.

In one embodiment, the microbe treated is a pathogenic bacterium.

In one embodiment, the microbe treated is a pathogenic fungus.

In one embodiment, a surface is treated selected from the group of human skin, plant skin, animal skin, or a device surface.

In one embodiment, a silver nanoparticle-embedded plant derived biomass waste particle with a concentration of silver of 0.1-500,000 mg/kg is described.

In one embodiment, the silver nanoparticle-embedded plant derived biomass waste particle wherein the biomass waste is cotton gin waste, cardboard, cotton gin motes, cotton seed hulls, rice straw, sugar cane bagasse or pine wood.

In one embodiment, the silver nanoparticle-embedded plant derived biomass waste particle having a concentration of silver is between 1-7 wt %.

In one embodiment, a method of treating a microbe by application of an effective amount of a composition comprising silver nanoparticle-embedded plant derived biomass waste particles is described.

In one embodiment, the biomass waste particle is derived from cotton gin waste, cardboard, cotton gin motes, cotton seed hulls, rice straw, sugar cane bagasse or pine wood.

The term cotton gin waste, also known as cotton gin trash, is the residual product generated during the cotton ginning process, which involves the separation and cleaning of cotton fibers from the seed bolls. This waste material consists of a mixture of lint, seeds, hulls, leaves, sticks, and dirt, leading to a composition that significantly differs from that of cotton fiber. While cotton fiber is predominantly composed of cellulose, cotton gin waste exhibits a heterogeneous nature, containing cellulose, hemicellulose, lignin, arabinan, and galactan, and various inorganic materials such as calcium, potassium, silica, manganese, zinc, iron, and nickel. In one aspect, the waste includes burs, bracts, sticks, seeds, and leaf fragments, which can be separated. In one aspect, the cotton fibers attached to the seeds, known as motes, can also be separated, but is not required.

In one aspect, the cotton gin waste nanofiber is produced by the steps of grinding cotton gin waste in a mill with about a 20-80 mesh sieve.

In one aspect, the cotton gin waste nanofiber is produced by treating the obtained powder sample after grinding with a 1-20 wt % aqueous NaOH solution to remove extractives. In one aspect, the cotton gin waste nanofiber is produced by treating the obtained powder sample after grinding with a 1-6 wt % aqueous NaOH solution to remove extractives.

In one aspect, the cotton gin waste nanofiber is produced by suspending the alkali-treated cotton gin waste in water and then high-shear mixing. In one aspect water or other fluid is used as an aid to processing. The high-shear mixer processes the solid material down to the desired particle size. In one aspect, the mixture is then pumped to the drying bed where the fluid is removed, leaving behind the granular product. In one aspect, the mixture is retained as a suspension or slurry.

In one aspect, the high shear mixing product of the step above is pumped through a ceramic interaction chamber. In one aspect, the high shear mixing product of the step above is pumped through a 200-1000 μm interaction chamber for 5-20 times. In one aspect, the high shear mixing product of the step above is pumped through a 100-300 μm interaction chamber 5-20 times. In one aspect, the high shear mixing product of the step above is pumped through a 50-200 μm interaction chamber 5-20 times,

In one aspect, the product of the step above is next pumped through an narrower interaction chamber. In one aspect, the product of the step above is pumped through a narrower interaction chamber added in series with a larger interaction chamber for optional additional passes with operating pressure set between 3 and 300 MPa.

In one aspect, the product of the step above is pumped through a diamond Z-shaped interaction chamber. In one aspect, the product of the step above is pumped through about a 87-150 μm diamond Z-shaped interaction chamber added in series with about a 200-300 μm auxiliary processing module for optional additional passes with operating pressure set between 3 and 300 MPa.

In one aspect, the product of the step above is pumped through about 90 μm diamond Z-shaped interaction chamber added in series with the 180-220 μm ceramic interaction chamber for an additional five passes with operating pressure set to about 200-220 MPa.

In one aspect, the product of the step above is used at a concentration of about 0.001-20 wt % and treated with an aqueous silver precursor solution.

In one aspect, the aqueous silver solution (0.1-100 mM) used to make silver nanoparticles described herein is silver nitrate. In one aspect, the aqueous silver solution used to make silver nanoparticles described herein is silver acetate, silver carbonate, silver sulfate, silver chloride, silver perchlorate, silver trifluoroacetate, or silver tetrafluoroborate.

In one aspect, the silver nanoparticle-embedded cotton gin waste nanofiber aerogel has a pore volume of 1 to 100 mm³/g. In one aspect, the silver nanoparticle-embedded cotton gin waste nanofiber aerogel has a pore volume of 20 to 50 mm³/g. In one aspect, the silver nanoparticle-embedded cotton gin waste nanofiber aerogel has a pore volume of about 30 mm³/g.

In one aspect, the silver nanoparticle-embedded cotton gin waste nanofiber has a surface area of 1 to 100 m²/g. In one aspect, the silver nanoparticle-embedded cotton gin waste nanofiber has a surface area of 2 to 50 m²/g. In one aspect, the silver nanoparticle-embedded cotton gin waste nanofiber has a surface area of 2 to 30 m²/g. In one aspect, the silver nanoparticle-embedded cotton gin waste nanofiber has a surface area of 9 m²/g.

The term dispersion is a dispersion within a host material with limited to no aggregation. In one aspect, a good dispersion of silver nanoparticles within a host material is obtained with limited to no aggregation of nanoparticles to each other observed.

The term “effective amount” or “effective dosage” is defined as an amount sufficient to achieve or at least partially achieve the desired effect.

Nanofibers are fibers with diameters in the nanometer range (typically, between 1 nm and 1 μm).

Composite refers to a material which is produced from two or more constituent materials exemplified herein by silver nanoparticles on a nanofiber matrix.

Nanoparticle-embedded are nanoparticles embedded in a polymer matrix exemplified by a nanoparticle such as a silver nanoparticle and a polymeric network. The polymer for example, can be derived from cotton gin waste in various embodiments described herein.

Mesh refers to the size of the spacing between evenly spaced holes in a standard sieve, and the number of a mesh indicates the size of the hole. For example, for 100 mesh, standard size dimension is 0.150 mm, 0.0059 inches or 150 microns.

Concentration of silver in this study is a mass concentration based on the dry weight of cotton gin waste matrix. The unit is wt %.

Extractives are the water and alkali soluble components of the cotton gin trash or other biomass material which can be removed by washing, filtration, or extraction using a suitable technique such as Soxhlet Extraction.

High-shear mixing is the process of a mixing in homogenizer. Homogenization is the process of making a substance uniform in terms of particle or globule size. In one embodiment, a microfluidizer is used, however any method of preparation of nanofibers can be employed that is known in the art. For example, wet-disk milling, ultrafine grinding with a supermasscoilloder and or impaction homogenizers that don't use a microfluidics chamber can be employed.

High-pressure homogenizer uses a piston pump to force a substrate through a very small adjustable aperture at high pressure within an interaction chamber. The interaction chamber may consist of an inlet, a high-shear zone or an impaction zone arranged in different geometries. For example, the interaction chamber may resemble a Z-shape or Y-shaped chamber where the solid in fluid enters through an aperture at one end and is passed through the inlet into narrow microchannels where the substrate experiences high shear. As the sample exits the channel it experiences cavitation and impact before flowing to the outlet. The combination of turbulence, shear, cavitation and impact subjects a substance to high energy forces reducing the substrate particle or globule down to sub-micron size.

Aperture width is defined as the cross-sectional diameter of the opening through which the fluid must pass through in the enclosed system. For example, in the microfluidics system the aperture can be in one aspect a 1010 μm, 550 μm, 425 μm, 400 μm, 300 μm, 250 μm, 200 μm, 150 μm, 100 μm, or 87 μm Z-shaped chamber; in one aspect the aperture may be a 125 μm or 75 μm Y-shaped chamber.

Application is a medicated or protective layer or material applied and dispersed by various means known in the art. Examples of applying the compositions described herein include application to tools, devices; animal or human skin; work surfaces, contact surfaces, etc. in domestic, medical, hospitality, recreational or other public and private environments. Further examples include agricultural work environments such as poultry, animal husbandry, and various forms of crop growing, horticulture and other processing activities and environments where inhibiting microbial growth is beneficial. Contacting or exposing objects with the antimicrobial compositions described herein (to reduce and/or kill bacteria for example) may occur by conventional methods such as spraying or dusting or dipping or immersion wherein the object is in contact with an antimicrobial powder, aerosol, emulsion or a solution for a certain period of time (e.g., about 120 seconds).

As used herein, the term “about” is defined as plus or minus ten percent of a recited value. For example, about 1.0 g means 0.9 g to 1.1 g.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

It will be obvious to those skilled in the art that such embodiments are provided by way of example only. Numerous variations, changes, and substitutions will occur to those skilled in the art without departing from the embodiments of the claims. Various alternatives to the embodiments of the claims described herein may be employed in practicing the use of compositions and methods of treatment described herein. It is intended that the included claims define the scope of the various compositions and methods of treatment described herein and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

Described below are abbreviations used herein.
CNF Cellulose nanofibers
CGT Cotton gin trash or waste,
Ag-CNF silver nanoparticle-embedded cellulose nanofibers
m=meter
mm=millimeter
nm=nanometer
g=gram
h=hour
w/v weight to volume, typically weight of solids to volume of solution
Ag NPs=silver nanoparticles
DI water deionized water, typically having a conductivity of 0.1 to 2 μS/cm

(microsiemens per centimeter) or a resistivity of 0.5 to 10 MΩ/cm (megaohms per centimeter)
wt % is a unit of concentration that expresses the mass of a component as a percentage of the total mass of a mixture or solution.
mT millitorr
DI water deionized water, typically having a conductivity of 0.1 to 2 μS/cm (microsiemens per centimeter) or a resistivity of 0.5 to 10 MΩ/cm (megaohms per centimeter)
TEM Transmission electron microscopy
AgNO₃ silver nitrate
rpm revolutions per minute
MPa megapascal
mM millimolar
CFU/mL colony forming units per milliliter
° C. degree Celsius
SEM Scanning electron microscopy XRD X-ray crystallography
MIC Minimum inhibitory concentration
TSA Tryptic Soy Broth
eV=electron volt
cps=count per second
keV=kiloelectron volt

### Examples

Having now generally described the compositions, methods of treatment and other embodiments described herein, the same will be better understood by reference to certain specific examples, which are included herein only to further illustrate the embodiments and are not intended to limit the scope of the same as defined by the claims.

Materials: Cotton gin motes, cotton gin trash, and cotton seed hulls were obtained from the USDA research facility in Stoneville, MS. Rice straw was a commercial package purchased from FloraCraft (Ludington, MI). Sugarcane bagasse was obtained from the USDA Commodity Utilization research unit in New Orleans, LA. Waste corrugated cardboard boxes (570 g/m²) were procured from Uline (Pleasant Prairie, WI). Untreated softwood pine (Pinus radiata) board was obtained from a local lumber yard (84 Lumber, St. Rose, LA).

CGT was sourced from the USDA Research Facility in Stoneville, Mississippi. Silver nitrate (AgNO₃, 99.9%) was purchased from J. T. Baker (Radnor, PA, USA), and sodium hydroxide (NaOH), methyl methacrylate, butyl methacrylate, methyl ethyl ketone, and benzoyl peroxide were purchased from Sigma-Aldrich (St. Louis, MO, USA). All chemicals were used as supplied, and deionized (DI) water was used as the solvent.

Preparation of cotton gin waste nanofiber: Cotton gin waste was ground in a Wiley mill (E3300, Eberbach Corp., Belleville, MI, USA) with a 20 mesh sieve. The obtained powder sample was treated for 2 h at 60° C. with a 4 wt % NaOH solution at a sample to liquor ratio of 1:20 (w/v) to remove extractives. The sample was then washed with deionized water until a neutral eluant (pH≈7) was achieved. The alkali-treated cotton gin waste was suspended in deionized water at approximately 0.5 wt % using an Ultra-Turrax® (T25, IKA Works, Inc., Wilmington, NC, USA) mechanical homogenizer. The slurry was then subjected to high-shear mixing with a Silverson L5M-A Laboratory Mixer equipped with a Square Hole High Shear Screen™ at 8,000 rpm for 10 min. The obtained lignocellulose slurry was then subjected to high-shearing forces using a high-pressure homogenizer (Microfluidizer M-110P, Microfluidics Corp., Newton, MA, USA). The suspension was pumped through one 200 μm ceramic interaction chamber five times, and then one 87 μm diamond Z-shaped interaction chamber was added in series with the 200 μm ceramic interaction chamber for an additional five passes. Operating pressure was set to 2100 MPa.

In situ synthesis of silver nanoparticles (Ag NPs) in cotton gin waste nanofiber: A 10 mL aqueous solution of AgNO₃ (12 or 58 mM) was added to a 50 mL suspension of cotton gin waste nanofiber. The resulting solution was heated at 100° C. for 30 min. After treatment, the nanofibers were washed with centrifugation multiple times in DI water.

Preparation of cellulose nanofiber suspension: CNF and Ag-CNF were dispersed in DI water at a concentration of 0.01 wt % and sonicated for 10 min.

Preparation of aerogel: A 5 mL aqueous suspension containing approximately 1 wt % of nanofibers was poured into a 20 mL disposable borosilicate vial. The vial was then placed into a Dewars vacuum bowl containing liquid nitrogen and frozen over ten minutes. The frozen vial was removed from the Dewars and immediately placed into a vacuum-safe flask and lyophilized using a VirTis Freezemobile 25EL freeze dryer (ATS, Warminster, PA USA) at 15 mT over 72 hr.

Ultraviolet-visible (UV-vis) spectra were collected using a UV-vis spectrometer (ISR-2600, Shimadzu) equipped with an integrating sphere unit. Absorbance spectra were collected in the wavelength range of 200-1100 nm.

Transmission electron microscopic (TEM) images were obtained using a JEM-2011 TEM (Jeol, Tokyo, Japan) operating at 120 kV. The nanofiber sample was placed in a BEEM capsule, and a mixture of methyl methacrylate and butyl methacrylate was added to the capsule. The mixture was then polymerized under UV light (CL-1000, UVP, Upland, CA, USA) for 30 min. A block of the embedded sample was cut into approximately 100 nm-thick slices using a PowerTome Ultramicrotome (Boeckeler Instruments, Tucson, AZ, USA). These thin films were placed on a carbon-film-coated copper grid. The size of silver nanoparticles was determined by analyzing TEM micrographs using Image J software.

The silver content in cotton gin waste nanofiber was measured using a graphite furnace atomic absorption spectrometer (240Z AA, Agilent, Santa Clara, CA, USA). Approximately 0.05 g of nanofibers was treated with 10 mL of 6 M nitric acid (Trace Metal Grade) and digested in a microwave digestion system (MARS 6, CEM Corporation, Matthews, NC, USA). The digest was diluted by weight 1:1000 and analyzed with an external calibration curve, which was obtained using silver single element standard (Agilent, Santa Clara, CA, USA). The average of three measurements was presented.

Energy-dispersive X-ray spectroscopy (EDS) analysis was conducted using a field-emission SEM (Quanta 3D FEG FIB/SEM, FEI) equipped with an EDS detector (Apollo XL, EDAX). The field emission gun was operated at an accelerating voltage of 5 kV and a gun current of 3.0 pA. The sample was mounted on a stub using double-sided carbon tape, and a platinum coating was sputtered onto the sample.

Scanning electron microscopic (SEM) images were obtained using a Phenom G6 ProX SEM (Nanoscience Instruments, Phoenix, AZ, USA) with an accelerating voltage of 10 kV. To prepare the sample, it was mounted on a stub using double-sided carbon tape, followed by sputtering a 5 nm-thick gold coating onto the sample.

Surface area and pore size of aerogel samples were measured using a Quantachrome NOVA 2200e adsorption system (Quantachrome Instruments, Boynton Beach, FL). Samples were out-gassed at 150° C. for 7 days under vacuum. Adsorption data were collected using 25 points on the nitrogen isotherm between 0.0499 and 0.9871 relative pressures at 77 K (−196° C.) and desorption data were collected using 12 points down to 0.04735 relative pressure at 77 K (−196° C.) (liquid nitrogen). The average of three measurements was presented.

X-ray diffraction (XRD) measurements were performed by the Shared Instrument Facility, Louisiana State University at room temperature using a Empyrean diffractometer (Malvern Panalytical, Malvern, United Kingdom) with Cu Kα-radiation (1.5418 Å). Data was collected with a spinning zero-background sample holder over the range of 5-80° 2θ, with a 0.013 step size and a radial divergence slit and nickel filter. A blank run of the sample holder was acquired prior to sample acquisition to capture the instrument background. The obtained powder patterns were analyzed using a pseudo-Voigt peak shape with the MAUD Rietveld program (Materials Analysis Using Diffraction, v. 2.93) for all powder patterns. 24, 25 The cellulose Iβ crystal information file (.cif) was supplied by Nishiyama et al. 26, 27 A cellulose II pattern with a crystallite size of 12 Å was used to model the amorphous phase. 28, 29 The crystalline phases for silver ICSD 604629 and silver oxide ICSD 173984 were modeled with the respective .cif files. 30, 31 The relative percentage of each phase was determined based on the area of the refined powder patterns after background subtraction, relative to the area of the total calculated pattern for each refinement. The crystallinity of the sample was calculated by taking the area of the refined cellulose Iβ pattern representing crystalline cellulose divided by the sum of the area for the crystalline and amorphous regions. The cellulose crystallite size perpendicular to the (hkl) lattice plane (Lhkl) was calculated using the following equation:

$${L}_{hkl} = \frac{0.9 λ}{{β}_{hkl} \times cos θ} \qquad ( 1 )$$

where the shape factor was taken as K=0.9, λ is the average Cu Kα-radiation wavelength (1.5418 Å), and β is the angular full width half maximum intensity (FWHM) in radians of the refined pattern, and θ is the scattering angle. The associated d-spacings were calculated from the refined unit cell dimensions.

The minimum inhibitory concentration tests against gram-positive Staphylococcus aureus (S. aureus) ATCC 6538 and gram-negative Pseudomonas aeruginosa (P. aeruginosa) ATCC 9027 were conducted by Situ Biosciences (Wheeling, IL, USA). For inoculum preparation, bacteria were propagated overnight in Tryptic Soy Broth. Prior to testing, the concentration of the test organisms is established using a spectrophotometer to measure optical density and then diluted to approximately 1×10² to 1×10³ CFU/ml. For testing, the 100 μL of the bacteria inoculum were pipetted into each test well of the 96-well plate. A serial 1:1-fold serial dilution was performed followed by 48 hours of incubation. The minimum inhibitory concentration was presented as the average value for 12 replicates.

The antibacterial properties against opportunistic bacterial pathogens, Pseudomonas aeruginosa (ATCC 9027) and Staphylococcus aureus (ATCC 6538) were tested at Situ Biosciences Microbial Product Test Laboratory (Wheeling, IL, USA). The minimum inhibitory concentration (MIC) was determined following the ISO 17025 standard. Bacterial cultures were grown in Tryptic Soy Broth (TSB) at 37° C. for 24 h and diluted to approximately 1×10³ CFU/mL in phosphate-buffered saline (PBS). Serial 1:1 eight-fold dilutions of the test sample were prepared in a 96-well plate, inoculated with 100 μl of the bacterial suspension, and incubated for 48 hours. The MIC value was obtained as the average concentration that inhibited bacterial growth across 12 replicates. To evaluate biocidal activity, bacterial suspensions were adjusted to approximately 1×10⁸ CFU/mL, and 10 μl of the inoculum was added to each of the three test sample replicates. Exposure durations of 1 h and 24 h were used, and the biocidal log reductions were calculated based on the difference in the common logarithms of pathogen populations between the control and test samples.

The antibacterial properties against foodborne bacterial pathogens, Salmonella Typhimurium (H3380) and Listeria monocytogenes (19116), were evaluated at the Microbial and Chemical Food Safety Research Unit (Wyndmoor, PA, USA). For determination of biocidal log reductions, bacterial strains were grown in 20 mL of TSB at 37° C. for 24 h (172 rpm), centrifuged (10,000×g, 5° C., 10 min), and the pellets resuspended in PBS. Inoculum levels were adjusted to ˜6 logs CFU/mL by serial dilution. For testing, 1.0 mL of each sample was mixed with 0.5 mL of bacterial suspension and incubated at 37° C. for 10 min or 24 h (exposure duration). Aliquots (100 μL) were spread on selective media (xylose lysine tergitol-4 for S. Typhimurium and modified Oxford agar for L. monocytogenes) and incubated at 37° C. for 24 h. Controls were included to quantify the initial inoculum, and pathogen reductions were calculated by subtracting CFU counts of test samples from controls.

A zone of inhibition assay was conducted by pipetting 50 μL of the test sample onto each of three TSA replicates inoculated with approximately 1×10⁶ CFU/mL. The plates were incubated at 37° C. for 24-48 h to evaluate the zones of bacterial growth inhibition.

Observation of the morphology of foodborne bacteria: 100 μL of cells were pipetted onto coverslips, incubated at 25° C. for 30 min, and chemically fixed with 2.5% glutaraldehyde. The fixative solution was decanted, and samples were washed twice with 0.1 M imidazole buffer. The samples were then dehydrated using a graded ethanol series (50%, 70%, 90%, and 100%) for 30 min at each concentration and subjected to critical point drying. The samples were sputter-coated with gold for 60 sec using an EMS/150R EMS (Hatfield, PA, USA), and these SEM images were obtained using an FEI Quanta 200F (Hillsboro, OR, USA). Control cells were included in the imaging process for comparison.

CGT contains naturally occurring reducing agents, such as lignin and hemicellulose, approximately 20 and 10%, respectively based on its dry weight. Hemicellulose, a branched polysaccharide, contains a variety of sugar units, including galactose. Its vicinal diols—two hydroxyl groups on adjacent carbon atoms—can undergo oxidation, forming dialdehyde compounds. The electrons released from the oxidation of diols reduce other chemical species. Lignin is a complex, heterogeneous polymer composed of phenolic units linked by various aliphatic and ether bonds. Its multiple functional groups, such as methoxy groups, hydroxyl groups, and aldehydes contribute to its redox activity. Additionally, the phenolic hydroxyl groups can participate in electron donation, further enhancing lignin's reducing capability.

The natural reducing agents in CNF were utilized to reduce Ag ions for in situ synthesizing Ag NPs. This approach offers two key advantages. First, it eliminates the need for external reducing agents, such as hydrazine or sodium borohydride, which are often toxic and environmentally hazardous. Second, it enables the direct embedding of Ag NPs within the CNF matrix. This embedding is highly advantageous as it creates a hybrid nanocomposite fiber, where the Ag NPs are stabilized, ensuring their long-term functionality.

The heat treatment of CNF in an aqueous solution induced the in situ synthesis of Ag NPs, changing the color of CNF from light brown to dark brown. This color change is attributed to the unique optical properties of Ag NPs, which arise from their surface plasmon resonance.

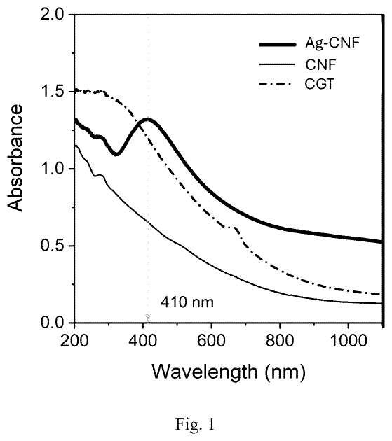

FIG. 1 shows the UV-vis spectra of cotton gin trash microparticles (CGT), CNF, and Ag-CNF. CGT and CNF showed no distinctive peaks, but Ag-CNF exhibited a strong surface plasmon resonance peak centered at 410 nm, characteristic of Ag NPs. This result suggests that the components in CNF, primarily hemicellulose and lignin, created a robust natural reducing system capable of inducing the in situ synthesis of Ag NPs. No external reducing or stabilizing agents were used in this process. Stabilizing agents are typically necessary to control particle growth and prevent aggregation, as NPs tend to cluster into large particles. The CNF matrix not only acted as an effective reducing agent, but also served as an active biotemplate to generate and stabilize NPs.

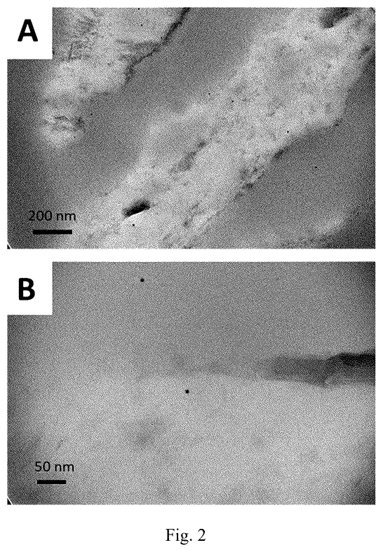

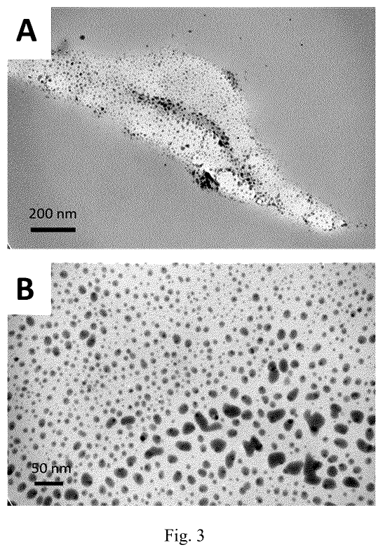

FIG. 2 shows TEM images of CNF at low (A) and high (B) magnifications. CNF did not show distinct structural details because of the low electron densities of its carbon-based organic components. FIG. 3 shows TEM images of Ag-CNF at low (A) and high (B) magnifications. These images show that CNF was filled with numerous spherical Ag NPs. The NPs were mostly well-separated from each other and relatively uniformly distributed throughout the matrix.

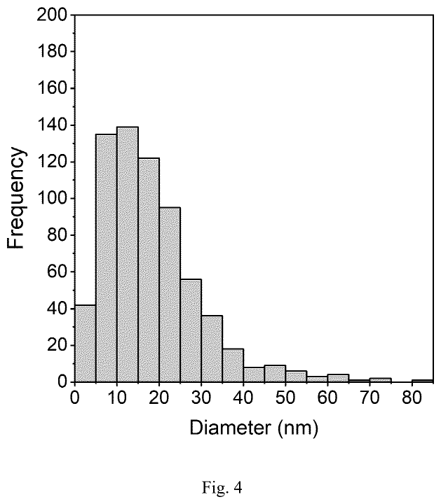

FIG. 4 shows the size distribution histogram of Ag NPs formed within CNF. The diameters were measured from the TEM images. The average diameter of Ag NPs was determined to be 18.2 nm.

In one aspect a method is described herein for preparing silver nanoparticle-embedded cotton gin waste microparticles wherein, the process for cotton gin waste microparticles to self-produce and self-embed silver nanoparticles wherein cotton gin waste microparticles do not requires the use of external reducing and stabilizing agents or dispersion processes for the silver nanoparticles.

In one aspect a method is described herein for preparing silver nanoparticle-embedded cellulose nanofibers wherein, the process for cellulose nanofibers to self-produce and self-embed silver nanoparticles wherein cellulose nanofibers do not require the use of external reducing and stabilizing agents or dispersion processes for the silver nanoparticles.

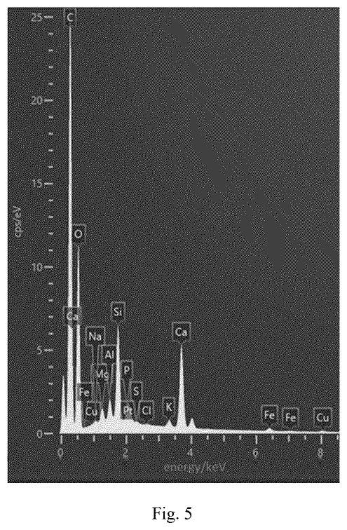

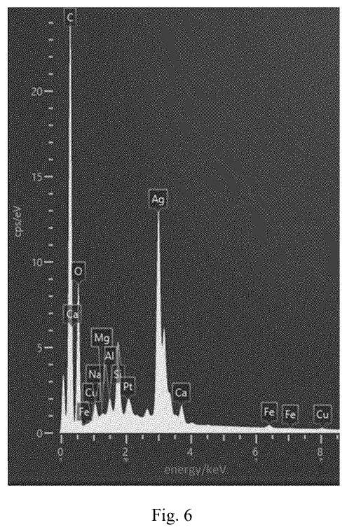

FIG. 5 shows the energy-dispersive X-ray spectrum (EDS) of CNF. FIG. 6 shows the energy-dispersive X-ray spectrum (EDS) of Ag-CNF. The spectrum of CNF shows that various inorganic elements are present in CNF. The spectrum of Ag-CNF shows strong signals of silver, which was absent in CNF.

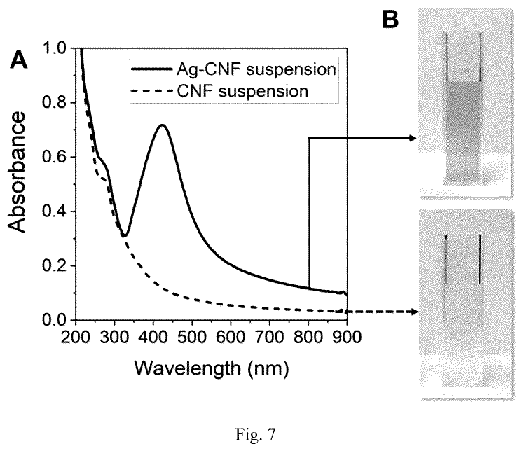

FIG. 7 shows the UV-vis spectra of CNF and Ag-CNF suspensions in DI water and their photographs. CNF suspension appeared nearly colorless, indicating its high dispersion and transparency. Ag-CNF suspension exhibited a vibrant yellow hue resulting from the surface plasmon resonance of Ag NPs. CNF suspension showed no distinctive peaks in its spectrum, while Ag-CNF exhibited a strong surface plasmon resonance peak centered at 423 nm, characteristic of Ag NPs.

The nano-in-nano structured Ag-CNF composite can be formulated into dispersions, coatings, films, etc. As a water-dispersible nanomaterial, it can be easily formulated and integrated for the fabrication of biomedical dressings, filtration membranes, and antimicrobial surface coatings. Additionally, its incorporation into flexible films offers potential applications in packaging and agricultural disease management.

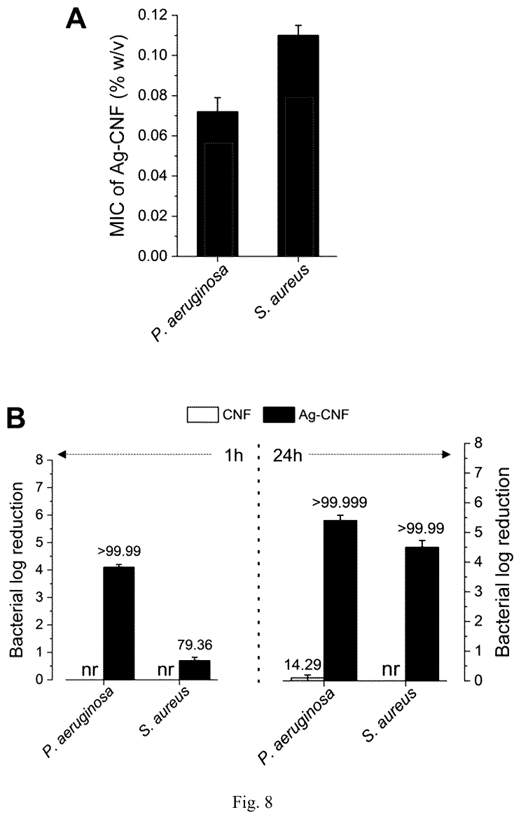

FIG. 8A shows that the MICs of Ag-CNF were determined to be 0.07% w/v for P. aeruginosa and 0.11% w/v for S. aureus. The antimicrobial efficacy at these low concentrations can be attributed to the unique “nano in nano” structure of the Ag-CNF composite. This configuration enhances the surface exposure of Ag NPs, which facilitates the release of Ag⁺ ions to promote greater interaction with bacterial cells. In contrast, CNF alone, at a concentration of 0.15% w/v, did not exhibit inhibitory effects on bacterial growth.

FIG. 8B shows the bactericidal efficacy of CNF and Ag-CNF at a concentration of 0.15% w/v against P. aeruginosa and S. aureus. Ag-CNF exhibited substantial bacterial reductions, achieving >5.4 Log CFU/mL for P. aeruginosa and >4.6 Log CFU/mL for S. aureus within 24 h. When the exposure duration was reduced to 1 h, the reductions were 4.1 Log CFU/mL for P. aeruginosa and 0.7 CFU/mL for S. aureus, corresponding to percent reductions of over 99.99% and 79.36%, respectively. CNF without embedded Ag NPs exhibited negligible antibacterial effects; after 1 h of exposure, no reduction in bacterial count was observed for either P. aeruginosa or S. aureus. Even after 24 h, CNF alone resulted in only a 14.3% reduction for P. aeruginosa and no measurable reduction for S. aureus.

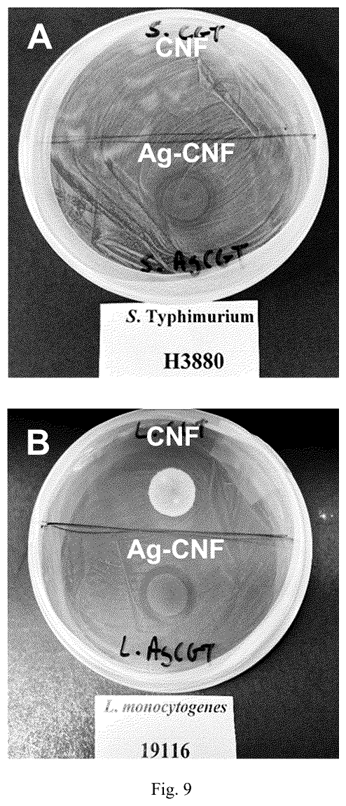

FIGS. 9A and 9B show the results of the zone of inhibition assay conducted to evaluate the antimicrobial activity of CNF and Ag-CNF against S. Typhimurium or L. monocytogenes, respectively, at a concentration of 0.15% w/v. The CNF exhibited no inhibitory effect on either S. Typhimurium or L. monocytogenes, as evidenced by the absence of inhibition zones. However, Ag-CNF demonstrated clear zones of inhibition, measuring 2.9±0.2 mm for L. monocytogenes and 2.3±0.3 mm for S. Typhimurium. The presence of inhibition zones indicates effective suppression of bacterial growth with a slightly greater efficacy observed against Gram-positive bacterium L. monocytogenes.

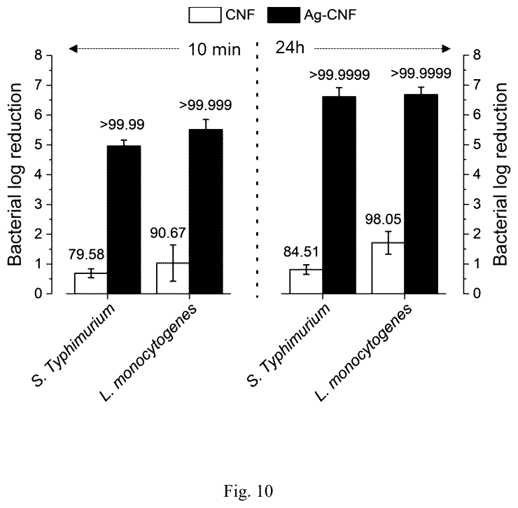

FIG. 10 shows the log reductions of S. Typhimurium and L. monocytogenes were measured after exposure to Ag-CNF and CNF for 10 min and 24 h. Within 24 h, Ag-CNF achieved inactivation levels exceeding 6.6 Log CFU/mL for both S. Typhimurium and L. monocytogenes. Within a 10 min-exposure, Ag-CNF resulted in reductions of >4.9 Log CFU/mL for S. Typhimurium and >5.5 Log CFU/mL for L. monocytogenes. CNF alone exhibited slight reductions in bacterial counts: with a 10 min-exposure, the log reductions were 0.69 Log CFU/mL for S. Typhimurium and 1.03 Log CFU/mL for L. monocytogenes. Increasing the exposure time to 24 h did not significantly influence the log reductions.

FIG. 11 shows SEM images of S. Typhimurium (A) unexposed, (B) exposed to CNF for 24 h, and (C) exposed to Ag-CNF for 24 h. FIG. 11C shows no NPs on the S. Typhimurium cells, and the integrity of their membranes is not significantly different from that of the unexposed S. Typhimurium cell. This result indicates that the Ag NPs embedded within the CNF did not leach out and mechanically attack the bacteria.

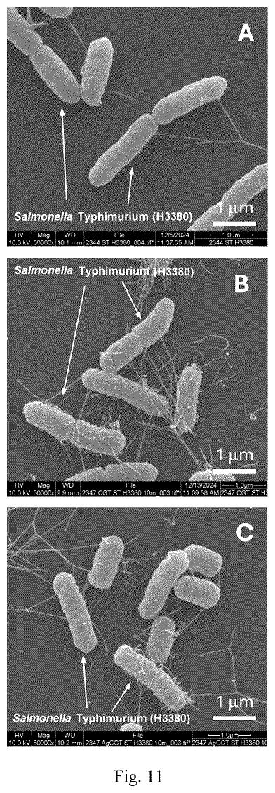

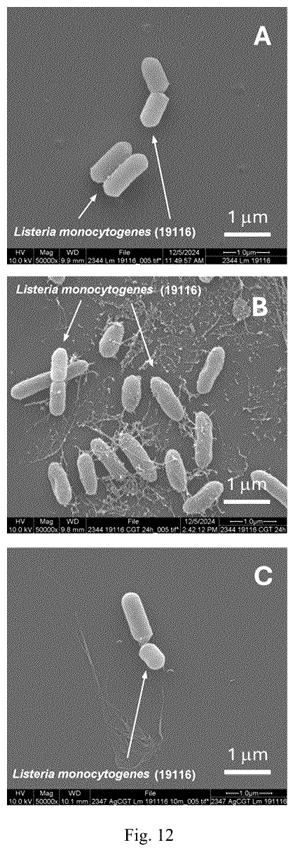

FIG. 12 shows SEM images of L. monocytogenes cells (A) unexposed, (B) exposed to CNF for 24 h, and (C) exposed to Ag-CNF for 24. FIG. 12C shows no NPs on the L. monocytogenes cells, and the integrity of their membranes is not significantly different from that of the unexposed L. monocytogenes cells. This result suggests that the Ag NPs remained embedded and stable within the CNF matrix, serving as a reservoir for the release of Ag ions. Ag ions are well known to exert antibacterial effects through the oxidative stress mechanism, generating reactive oxygen species that damage intracellular components.

The concentration of silver in cotton gin waste nanofiber was measured to be 18.7±6.7 wt %.

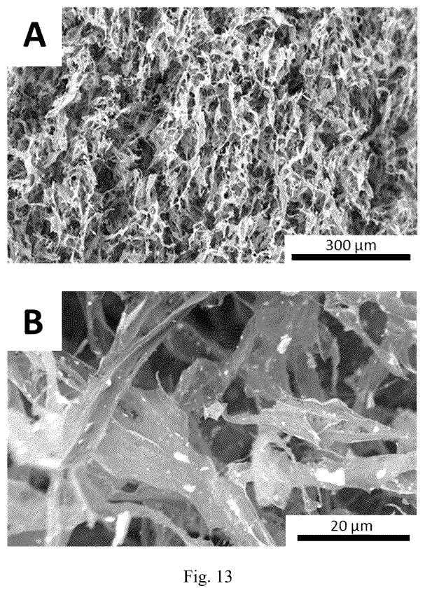

FIG. 13 shows SEM images of the aerogel fabricated with CNF at (A) low and (B) high magnifications. FIG. 14 shows SEM images of the aerogel fabricated with Ag-CNF at (A) low and (B) high magnifications. The comparison of these images shows that Ag NPs embedded within CNF did not significantly influence the fabrication of aerogel and its structure.

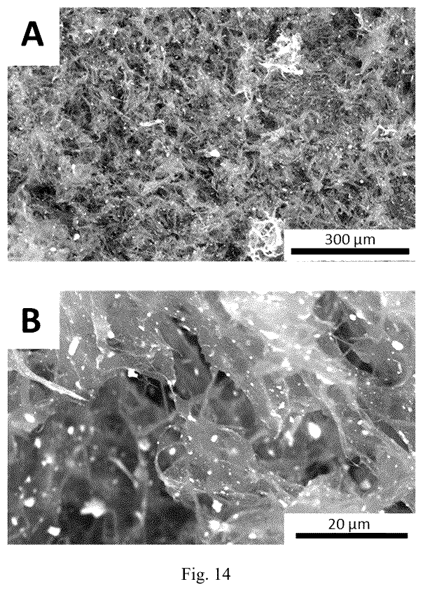

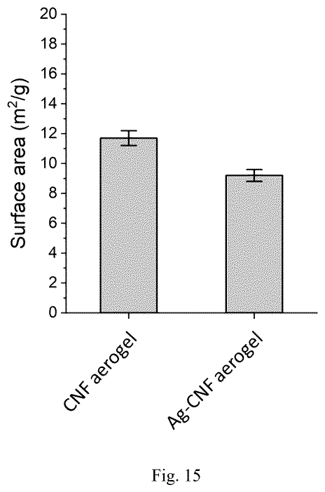

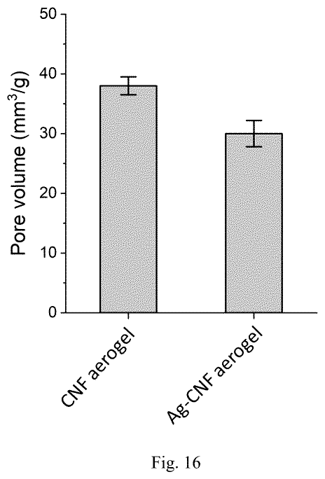

FIG. 15 shows the surface areas of aerogels fabricated with CNF and Ag-CNF. The surface area of Ag-CNF aerogel was slightly smaller than that of CNF aerogel. FIG. 10 shows the pore volumes of aerogels fabricated with CNF and Ag-CNF. The pore volume of Ag-CNF was slightly smaller than that of CNF-aerogel.

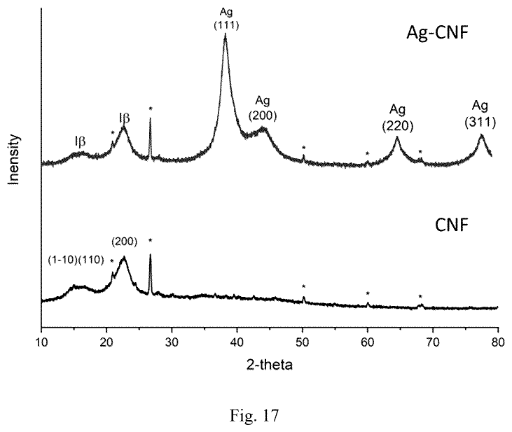

FIG. 17 shows XRD pattern of CNF and Ag-CNF. CNF exhibited a typical cellulose Iβ pattern with major reflections at approximately 14.9°, 16.8°, and 22.5° 2θ, attributed to the reflections about the (1-10), (110) and (200) lattice planes. CNF also contains several metallic salt impurities and contains 35% crystalline cellulose and 65% amorphous cellulose. Ag-CNF has a similar cellulose crystal size and structure with CNF. The XRD pattern of Ag-CNF indicates formation of cubic phase metallic Ag NPs with lattice constant of a=b=c=4.073.

Preparation of biomass waste microparticles from cardboard, gin motes, seed hulls, rice straw sugar cane bagasse and pine wood: Biomass waste samples were ground in a Wiley mill (E3300, Eberbach Corp., Belleville, MI, USA) with a 20-mesh sieve.

In situ synthesis of silver nanoparticles (Ag NPs) in biomass waste microparticles: Biomass microparticles (0.5 g) were suspended in 25 mL of an aqueous solution of AgNO₃ (25 mM). The resulting solution was heated at 100° C. for 30 min. After treatment, the sample was rinsed with DI water multiple times using centrifuge (10,000×g, 10 min). The Ag NP-embedded biomass waste particles were collected by filtration using a Buchner funnel with a fine porosity fritted disc and washed with water (3×50 mL). The obtained solids were oven-dried (40° C.) for 48 h.

In situ synthesis of silver nanoparticles (Ag NPs) in biomass waste particles under alkali: Biomass waste particles (0.5 g) were suspended in 25 mL of an aqueous solution of AgNO₃ (25 mM). To this was added 0.5 mL aqueous NaOH (0.25 N) to give a final concentration of about 5 mM NaOH. The resulting solution was heated at 100° C. for 30 min. After treatment, the sample was rinsed with DI water multiple times using centrifuge (10,000×g, 10 min). The Ag NP-embedded biomass waste particles were collected by filtration using a Buchner funnel with a fine porosity fritted disc and washed with water (3×50 mL). The obtained solids were oven-dried (40° C.) for 48 h.

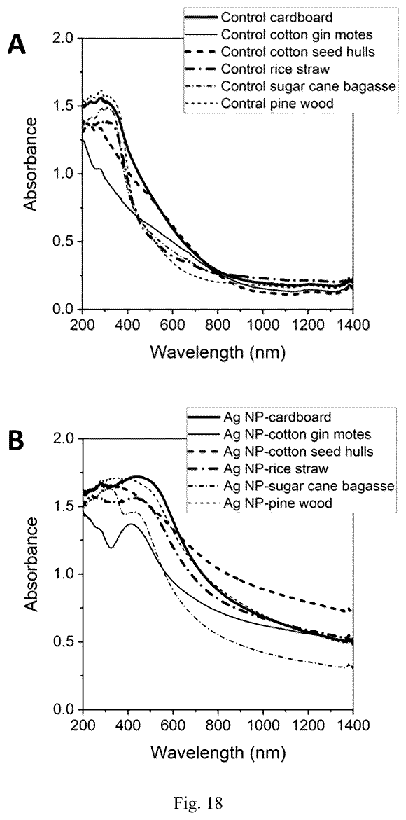

FIG. 18 shows the UV-vis spectra of (A) control biomass microparticle and (B) Ag NP-biomass particles. After the treatment, all biomass particles exhibited new strong absorbance peaks centered at 410-430 nm, indicating the in situ synthesis of Ag NPs by biomass microparticles.

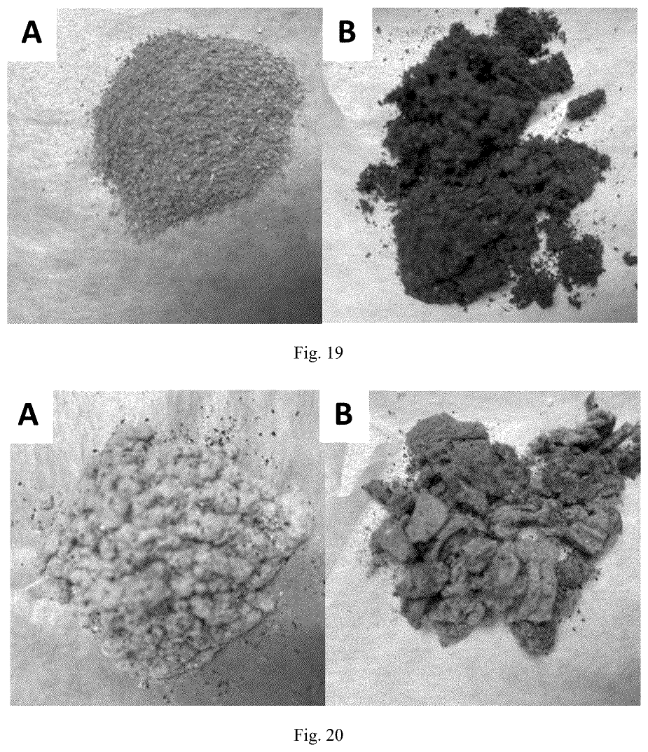

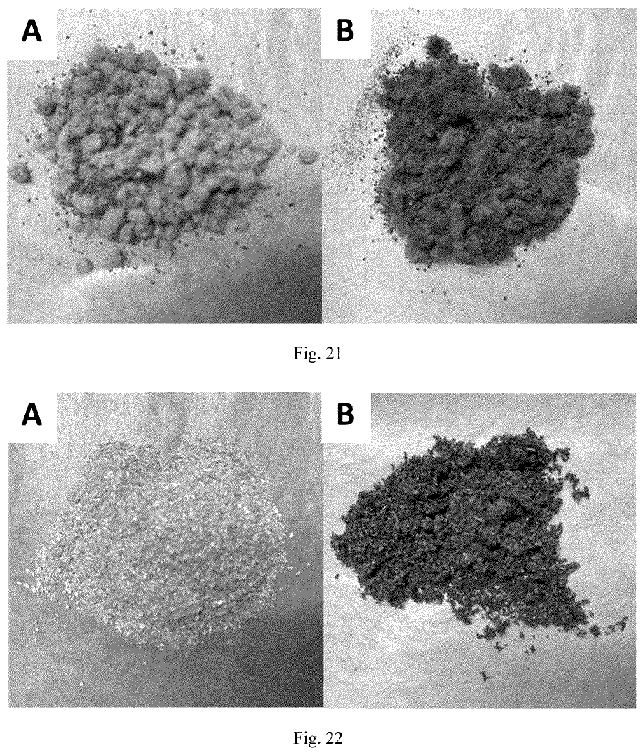

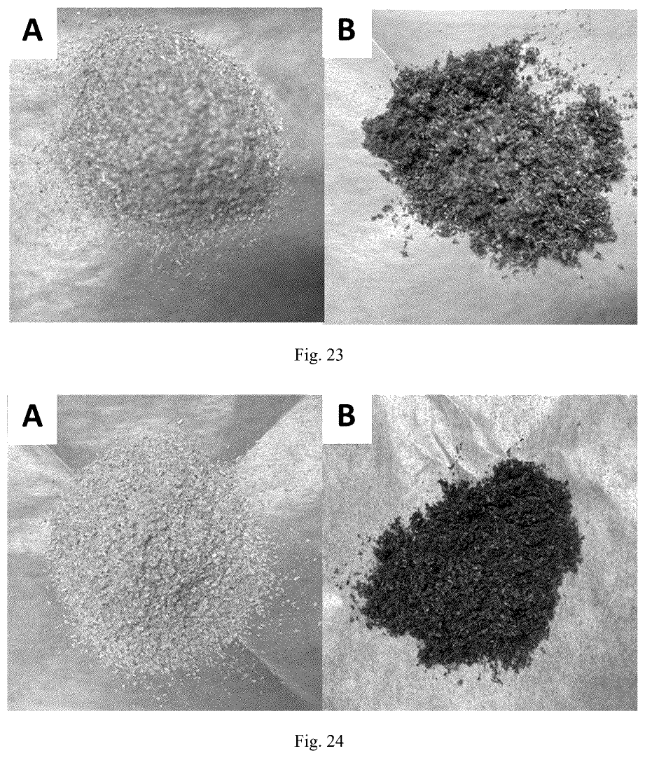

FIGS. 19-24 show the photographic images collected for cardboard, cotton gin motes, cotton seed hulls, rice straw, sugar cane bagasse, and pine wood microparticles, respectively, (A) before and (B) after the treatment. The in-situ synthesis of Ag NPs introduced darker colors to all biomass microparticles due to the surface plasmon resonance of Ag NPs.

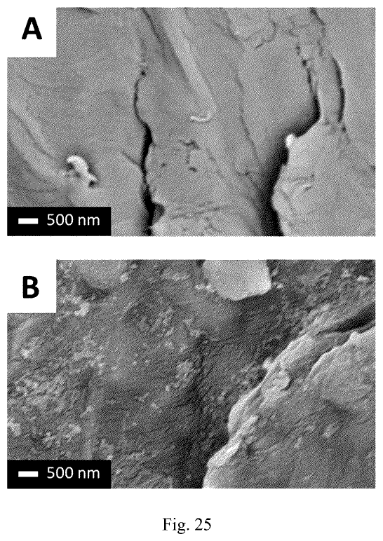

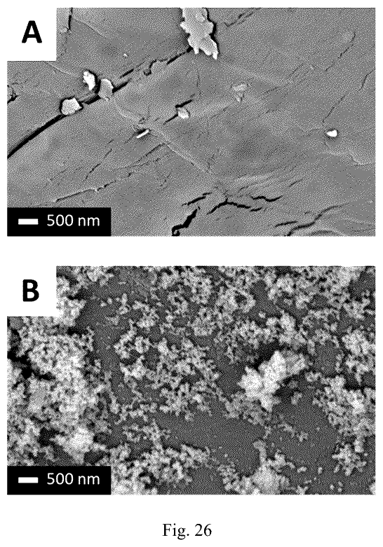

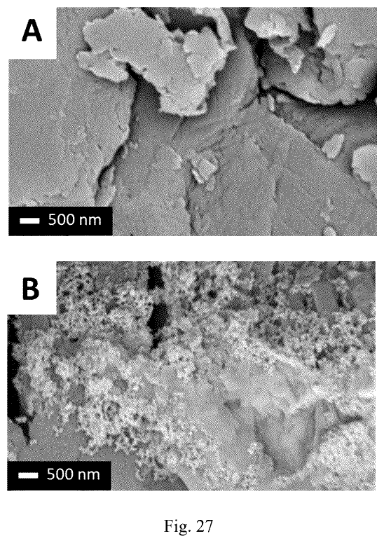

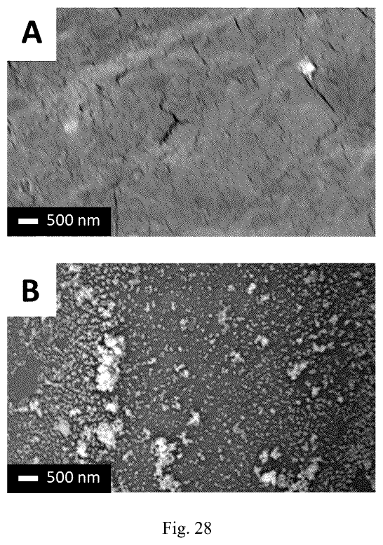

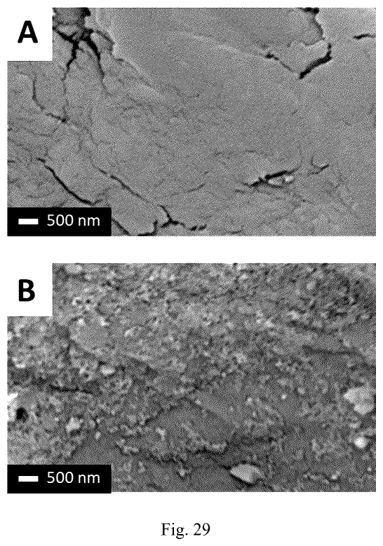

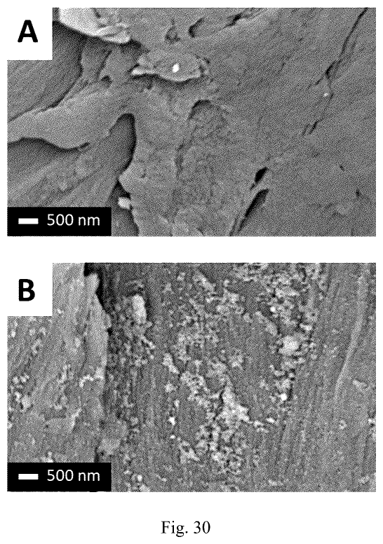

FIGS. 25-30 show SEM images of cardboard, cotton gin motes, cotton seed hulls, rice straw, sugar cane bagasse, and pine wood particles, respectively before (top) and after (bottom) the treatment. The comparison with the control samples (before treatment), all treated samples showed the formation of Ag NPs.

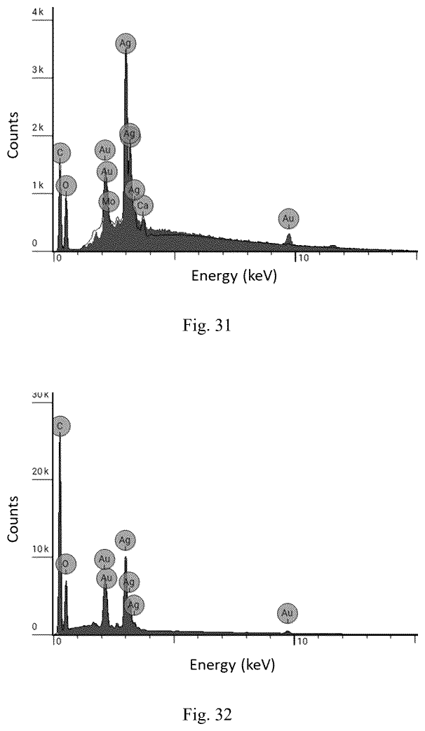

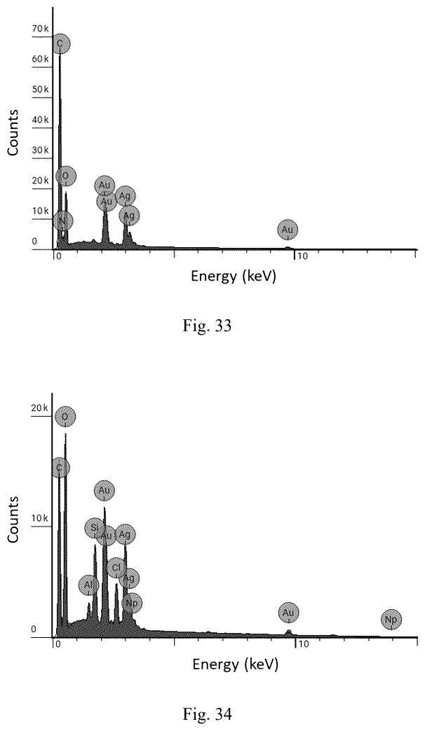

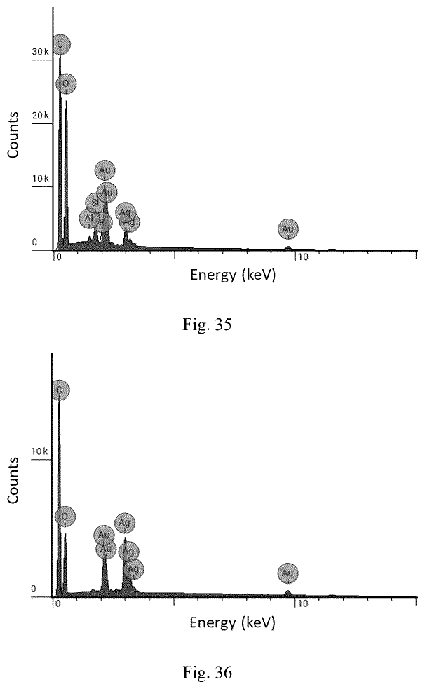

FIGS. 31-36 show the energy-dispersive X-ray spectra (EDS) of the treated cardboard, cotton gin motes, cotton seed hulls, rice straw, sugar cane bagasse, and pine wood particles, respectively. All spectra show strong signals for silver.

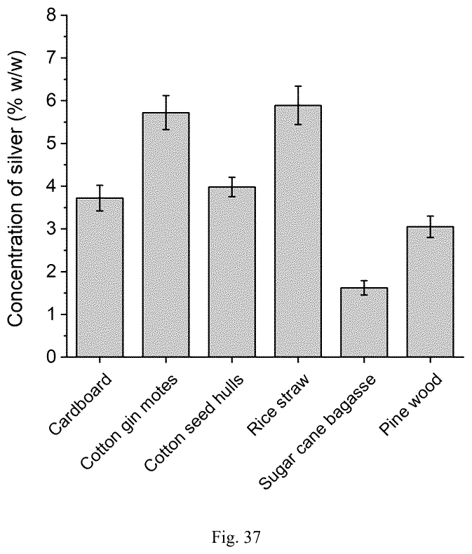

FIG. 37 shows the concentration of silver in various biomass microparticles after the treatment. The concentration varied from 1.6-5.9 wt % between the different biomass waste samples.

We claim:

1. A silver nanoparticle-embedded cellulosic biomass nanocomposite with a concentration of silver of 0.1-500,000 mg/kg.

2. The silver nanoparticle-embedded cellulosic biomass nanofiber composite of claim 1 wherein the biomass source is cotton gin waste.

3. The silver nanoparticle-embedded cotton gin waste nanofiber composite of claim 2 with a concentration of silver is between 0.1-30 wt %.

4. The silver nanoparticle-embedded cotton gin waste nanofiber of claim 2 with a concentration of silver is about 19 wt %.

5. The silver nanoparticle-embedded cotton gin waste nanofiber composite of claim 2 with silver nanoparticles having a particle size between 1 nm and 150 nm in the cotton gin waste nanofiber matrix.

6. The silver nanoparticle-embedded cotton gin waste nanofiber composite of claim 2 with silver nanoparticles having size of between 3 nm and 50 nm.

7. The silver nanoparticle-embedded cotton gin waste nanofiber composite of claim 2 with silver nanoparticles having average size of about 18 nm.

8. The silver nanoparticle-embedded cotton gin waste nanofiber composite of claim 2 with silver nanoparticles having uniformity in size and distribution in the cotton gin waste nanofiber.

9. A method of producing silver nanoparticle-embedded cotton gin waste nanofiber composite comprising the steps of:
a) preparing a cotton gin waste nanofiber,
b) treating of cotton gin waste nanofiber suspension with an aqueous silver precursor solution to form the silver nanoparticle-embedded cotton gin waste nanofiber.

10. The method of claim 8 where the cotton gin waste nanofiber composite is produced by the steps of:
grinding cotton gin waste in a mill to a size of 20 to 80 mesh sieve,
treating the obtained powder sample with a fluid to remove extractives,
washing with water,
suspending the alkali-treated cotton gin waste in water and then high-shear mixing,
homogenizing the lignocellulose slurry with high-shearing forces using a high-pressure homogenizer,
pumping through a 200-1000 μm interaction chamber five times,
pumping through a second 50-500 μm interaction chamber with a narrower aperture added in series with a 200-1000 μm interaction chamber for one or more passes with operating pressure set to about 200-300 MPa.

11. The method of claim 9 where the operating pressure was set to about 210 MPa.

12. The method of claim 9 where the aqueous silver precursor solution comprises silver nitrate.

13. The method of claim 9 of further comprising the step of washing to remove unreacted silver nitrate precursor to form said silver nanoparticle-embedded cotton gin waste nanofiber.

14. The method of claim 9 where the interaction chamber is Z-shaped and about 20-200 μm.

15. The method of claim 14 where the Z-shaped interaction chamber is about 90 μm.

16. The method of claim 9 where the fluid is a solution to remove extractives which is alkaline.

17. The method of claim 9 where the fluid to remove extractives is an aqueous NaOH solution.

18. A silver nanoparticle-embedded cotton gin waste nanofiber aerogel composition.

19. The silver nanoparticle aerogel of claim 18 with a pore volume of 1 to 100 mm³/g

20. The silver nanoparticle aerogel of claim 18 with a pore volume of 30 mm³/g

21. The silver nanoparticle aerogel of claim 18 with a surface area of 1 to 100 m²/g.

22. The silver nanoparticle aerogel of claim 18 with a surface area of about 9 m²/g.

23. The method of claim 18 with a concentration of silver nanoparticles of 0.1-500,000 mg/kg in the aerogel composition.

24. A method of treating a microbe by application of an effective amount of a composition comprising a silver nanoparticle-embedded cotton gin waste nanofiber composite.

25. The method of claim 24 wherein treating results in microbial growth inhibition.

26. The method of claim 24 wherein the microbe is a pathogenic bacterium.

27. The method of claim 24 wherein the microbe is a pathogenic fungus.

28. The method of claim 24 wherein the surface treated is selected from the group consisting of human skin, plant skin, animal skin, or a device surface.

29. A silver nanoparticle-embedded plant derived biomass waste particle with a concentration of silver of 0.1-500,000 mg/kg.

30. The silver nanoparticle-embedded plant derived biomass waste particle of claim 29 wherein the biomass waste is cotton gin waste, cardboard, cotton gin motes, cotton seed hulls, rice straw, sugar cane bagasse or pine wood.

31. The silver nanoparticle-embedded plant derived biomass waste particle of claim 29 wherein a concentration of silver is between 1-7 wt %.

32. A method of treating a microbe by application of an effective amount of a composition comprising silver nanoparticle-embedded plant derived biomass waste particles.

33. The method of claim 32 wherein the biomass waste particle is a cotton gin waste, cardboard, cotton gin motes, cotton seed hulls, rice straw, sugar cane bagasse or pine wood waste particle composition.
